(19) United States
(12) Patent Application Publication
(10) Pub. No.: US 2020/0315592 A1
(43) Pub. Date: Oct. 8, 2020

(54) WIRELESS ULTRASOUND ARCHITECTURES

(71) Applicants: Hamid Soleimani, Guilford, CT (US); Graham Peyton, Madison, CT (US); Nevada J. Sanchez, Guilford, CT (US)

(72) Inventors: Hamid Soleimani, Guilford, CT (US); Graham Peyton, Madison, CT (US); Nevada J. Sanchez, Guilford, CT (US)

(73) Assignee: Butterfly Network, Inc., Guilford, CT (US)

(21) Appl. No.: 16/836,497

(22) Filed: Mar. 31, 2020

Related U.S. Application Data
(60) Provisional application No. 62/830,325, filed on Apr. 5, 2019.

(51) Int. Cl. A61B 8/00

(52) U.S. Cl. CPC A61B 8/565; A61B 8/4483; A61B 8/4472

(57) ABSTRACT

An ultrasound probe is provided including one or more ultrasound transducers configured to perform ultrasound imaging, a first logic unit configured to receive ultrasound data from the one or more ultrasound transducers, and a second logic unit coupled to the first logic unit and configured to transmit the ultrasound data wirelessly via a radio module. An ultrasound device is provided configured for removably coupling to an auxiliary module to transmit ultrasound wirelessly via the auxiliary module.

### CROSS-REFERENCE TO RELATED APPLICATIONS

This application claims the benefit under 35 U.S.C. § 119(e) of U.S. Patent Application Ser. No. 62/830,325, filed Apr. 5, 2019, under Attorney Docket No. B1348.70123US00, and entitled “WIRELESS ULTRASOUND ARCHITECTURES,” which is hereby incorporated herein by reference in its entirety.

### FIELD OF THE DISCLOSURE

The present application relates to an ultrasound device that can perform ultrasound imaging and transmit ultrasound data wirelessly to one or more other devices.

### BACKGROUND

Ultrasound imaging systems typically include an ultrasound probe connected to a host by an analog cable. The ultrasound probe is controlled by the host to emit and receive ultrasound signals. The received ultrasound signals are processed to generate an ultrasound image.

### SUMMARY OF THE DISCLOSURE

Aspects of the present application relate to ultrasound device architectures for wirelessly transmitting ultrasound data to one or more other devices.

Some aspects relate to an ultrasound device configured for removably coupling to an auxiliary component to transmit ultrasound data wirelessly via the auxiliary component.

In some embodiments, the ultrasound device may include an ultrasound probe.

In some embodiments, the ultrasound device may be further configured to transmit the ultrasound data wirelessly via the auxiliary component to a second device.

In some embodiments, the ultrasound device may further comprise one or more transducers configured to perform ultrasound imaging, and processing circuitry configured to generate ultrasound data for transmitting wirelessly via the auxiliary component.

In some embodiments, the one or more transducers may comprise capacitive micromachined ultrasound transducers (CMUTs).

In some embodiments, the processing circuitry may comprise one or more groups of digital logic gates.

In some embodiments, the one or more groups of digital logic gates may comprise one or more field programmable gate arrays (FPGAs).

In some embodiments, the one or more groups of digital logic gates may comprise one or more application specific integrated circuits (ASICs).

In some embodiments, the one or more groups of digital logic gates may comprise a reduced instruction set computing (RISC) processor.

In some embodiments, the RISC processor may be an advanced RISC machine (ARM) processor.

In some embodiments, the processing circuitry may be configured to transmit the ultrasound data to the auxiliary component using a universal serial bus (USB) protocol.

In some embodiments, the ultrasound device may further comprise a USB connector configured for removably coupling to a USB connector of the auxiliary component.

In some embodiments, the processing circuitry may be configured to communicate with a radio module of the auxiliary component.

In some embodiments, the radio module may include a WiFi module.

In some embodiments, the processing circuitry may be configured to communicate with a processor of the auxiliary component.

In some embodiments, the ultrasound device may further comprise a memory module coupled to the processing circuitry.

In some embodiments, the ultrasound device may further comprise a first substrate having mounted thereon the one or more transducers, and a second substrate having mounted thereon the processing circuitry.

Some aspects relate to an ultrasound device comprising one or more ultrasound transducers configured to perform ultrasound imaging, a first logic unit configured to receive ultrasound data from the one or more ultrasound transducers, and a second logic unit coupled to the first logic unit and configured to transmit the ultrasound data wirelessly via a radio module.

In some embodiments, the first logic unit may comprise a first group of digital logic gates.

In some embodiments, the first group of digital logic gates may comprise a field programmable gate array (FPGA).

In some embodiments, the first group of digital logic gates may comprise an application specific integrated circuit (ASIC).

In some embodiments, the first logic unit and the second logic unit may be configured to communicate using a general programmable interface (GPIF) protocol.

In some embodiments, the second logic unit may comprise a second group of digital logic gates.

In some embodiments, the second group of digital logic gates may comprise a processor.

In some embodiments, the processor may comprise a reduced instruction set computing (RISC) processor.

In some embodiments, the RISC processor may comprise an advanced RISC machine (ARM) processor.

In some embodiments, the processor may be configured to run an operating system.

In some embodiments, the operating system may be Unix-based.

In some embodiments, the processor may be configured to manage wireless networking protocols for transmitting the ultrasound data wirelessly via the radio module.

In some embodiments, the radio module may include a WiFi module.

In some embodiments, the ultrasound device may further comprise a memory module coupled to the second logic unit.

### BRIEF DESCRIPTION OF THE DRAWINGS

Various aspects and embodiments of the disclosed technology will be described with reference to the following figures. It should be appreciated that the figures are not necessarily drawn to scale. Items appearing in multiple figures are indicated by the same reference number in all the figures in which they appear.

FIG. 1 is a block diagram of an illustrative example of a wireless ultrasound system including an ultrasound device and an associated device, in accordance with some embodiments of the technology described herein.

FIG. 2A is a block diagram illustrating an exemplary embodiment of processing circuitry for wireless transmission of ultrasound data, in accordance with some embodiments of the technology described herein.

FIG. 2B is a block diagram illustrating an exemplary embodiment of processing circuitry for a wireless ultrasound device removably coupled to an auxiliary component, in accordance with some embodiments of the technology described herein.

FIG. 2C is a block diagram illustrating an exemplary embodiment of processing circuitry for wireless transmission of ultrasound data, including multiple logic units, in accordance with some embodiments of the technology described herein.

FIG. 3A is a diagram of an exemplary wireless ultrasound system illustrating how a handheld wireless ultrasound device may be used to image a subject and wirelessly communicate with an associated device, in accordance with some embodiments of the technology described herein.

FIG. 3B is a diagram of an exemplary wireless ultrasound system illustrating how a wireless ultrasound pill may wirelessly communicate with an associated device, in accordance with some embodiments of the technology described herein.

FIG. 4A is a perspective view of the handheld wireless ultrasound device of FIG. 3A.

FIG. 4B illustrates an exemplary handheld wireless ultrasound device removably coupled to an auxiliary component, in accordance with the embodiment of FIG. 2B.

FIG. 5 illustrates a patch including a wireless ultrasound device, in accordance with some embodiments of the technology described herein.

FIG. 6 is a block diagram of an illustrative example of a wireless ultrasound device, in accordance with some embodiments of the technology described herein.

FIG. 7 is a block diagram illustrating how, in some embodiments, the transmit (TX) circuitry and the receive (RX) circuitry for a given transducer element of a wireless ultrasound device may be used either to energize the element to emit an ultrasonic pulse, or to receive and process a signal from the element representing an ultrasonic pulse sensed by the transducer element, in accordance with some embodiments of the technology described herein.

FIG. 8 shows an illustrative arrangement of ultrasonic transducers integrated with the substrate of a wireless ultrasound device, in accordance with some embodiments of the technology described herein.

FIG. 9 is a cross-sectional view of a device including a CMOS wafer integrated with a substrate having sealed cavities, in accordance with some embodiments of the technology described herein.

### DETAILED DESCRIPTION

The present disclosure describes aspects of an ultrasound device capable of transmitting ultrasound data wirelessly to another device to create ultrasound images using the ultrasound data. In a first embodiment, a wireless ultrasound device may be removably coupled to an auxiliary component which includes a radio module, such that ultrasound data may be transmitted to another device via the auxiliary component. In a second embodiment, a wireless ultrasound device including one or more ultrasound transducers may also include a first logic unit coupled to the ultrasound transducer(s) for receiving the ultrasound data, and a second logic unit coupled to the first logic unit to transmit the ultrasound data wirelessly to another device via a radio module.

The inventors have recognized that conventional ultrasound devices (e.g., ultrasound probes) are limited because they require a hardwired connection to other devices which create ultrasound images using data from the ultrasound device. For example, some conventional ultrasound devices connect to other devices by way of a wire such as in an electrical cable. Such wires and/or cables are inconvenient in that they can obstruct free movement of the ultrasound device around a patient. Further, they can become tangled around themselves or around parts of a patient. These cables are usually required to provide power to operate the ultrasound device and/or to facilitate the high volume of ultrasound data captured by the ultrasound device.

By contrast, the ultrasound devices developed by the inventors and described herein are configured for wirelessly transmitting ultrasound data such that wires, cables, cords, etc. may be eliminated entirely. In some embodiments, ultrasound devices developed by the inventors may be configured for removably coupling to an auxiliary component to wirelessly transmit the ultrasound data. For example, the ultrasound device may include a universal serial bus (USB) port, and the auxiliary component may include a USB connector, such that the ultrasound device may pass the ultrasound data to the auxiliary component via USB to be transmitted wirelessly to an associated device by a radio module of the auxiliary component. In some embodiments, the ultrasound device may include one or more ultrasound transducers, a radio module, and one or more logic units coupled between the ultrasound transducer(s) and the radio module to obtain the ultrasound data and to transmit the data wirelessly to an associated device. For example, the other device may use the ultrasound data to create ultrasound images. In accordance with various embodiments, the associated device receiving the ultrasound data may include a computer, a tablet computer, a mobile phone, a wearable device such as a smart watch, or any other suitable computing device capable of receiving wireless data transmission.

In some embodiments, the ultrasound devices described herein can leverage computerized techniques developed by the assignee to provide wireless communication links that facilitate transmission of large volumes of ultrasound data (e.g., using the often limited bandwidth afforded by wireless systems). In some embodiments, ultrasound devices described herein may be capable of data transfer rates as high as 150 Mbps via transmission control protocol (TCP), 170 Mbps via user datagram protocol (UDP). In addition, ultrasound devices described herein may operate with low enough power consumption, including while performing ultrasound imaging and while transmitting ultrasound data wirelessly, to be powered by an internal and/or attached power source, such as one or more batteries. For example, in some embodiments, ultrasound devices may require as little power as 1.5 W for data transmission. In some embodiments, the power source may include one or more rechargeable batteries. In some embodiments, the included power source may provide adequate power to operate the ultrasound device while maintaining a small form factor, such as an ultrasound pill that can be swallowed by a patient, as described further herein.

An ultrasound device may be implemented in any of a variety of physical configurations including, for example, as a part of an internal imaging device, such as a pill to be swallowed by a subject or a pill mounted on an end of a scope or catheter, as part of a handheld probe, as part of a patch configured to be affixed to the subject, and/or the like. The architectures described herein can be used for any of such various configurations, as described further herein.

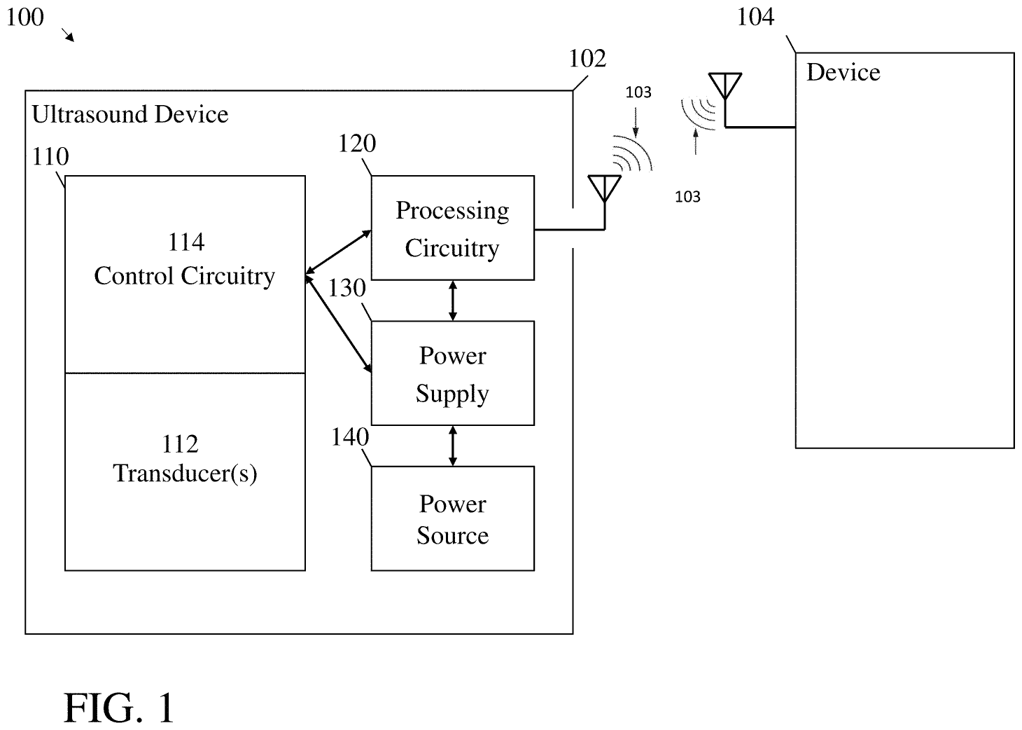

Turning to the Figures, FIG. 1 is a block diagram of exemplary wireless ultrasound system 100, in accordance with some embodiments of the technology described herein. In the illustrative embodiment of FIG. 1, system 100 includes ultrasound device 102 and associated device 104. Device 102 includes transducer circuitry 110, as well as processing circuitry 120 for wirelessly transmitting ultrasound data from the transducer(s) 112, and power supply 130 to provide power to transducer(s) 112, control circuitry 114, and/or processing circuitry 120 from power source 140.

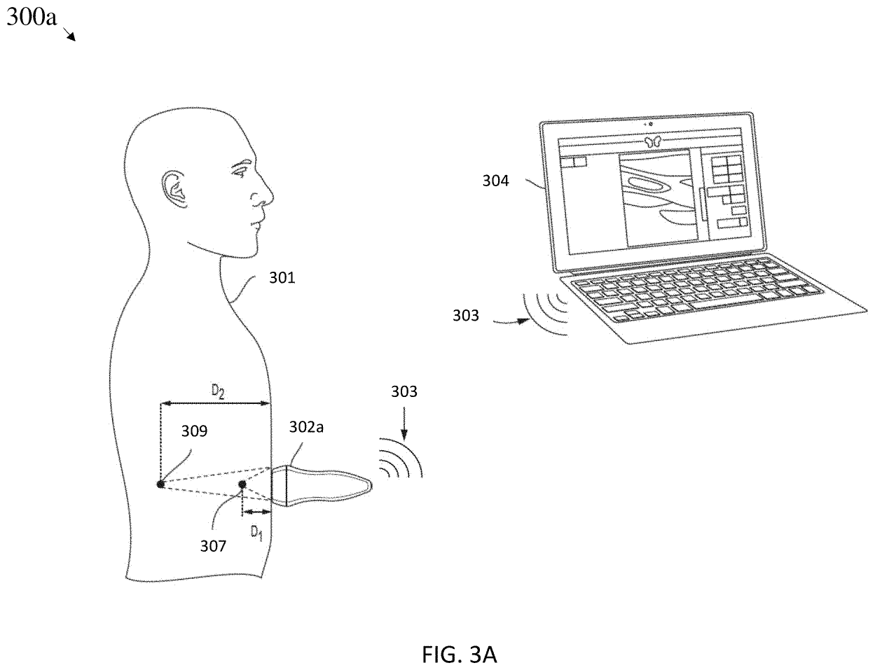

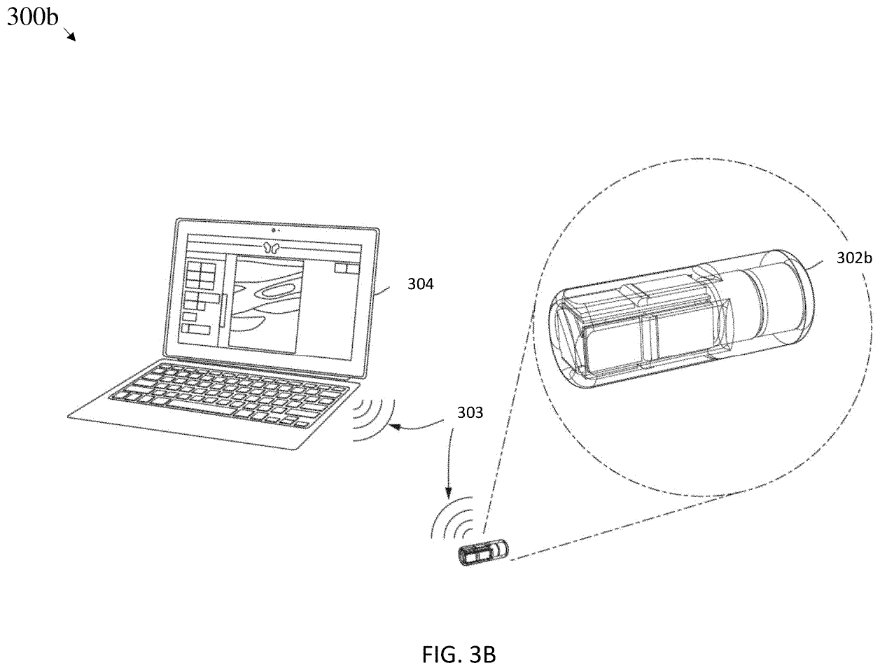

Device 102 and associated device 104 are wirelessly coupled by connection 103. In accordance with various embodiments, connection 103 may include any suitable wireless connection. In some embodiments, the wireless connection includes one or more short- or long-range connections. Exemplary short-range connections include, ZigBee, Bluetooth (BT), Bluetooth Low Energy (BLE), and Near-Field Communication (NFC). Long-range communication platforms can include, for example, Wi-Fi and Cellular. In instances where connection 103 is a WiFi connection, device 102 and associated device 104 may communicate by way of one or more routers or switches (not shown), for example if device 102 and associated device 104 are located at some distance from one another, such as in separate rooms. Exemplary devices configured in the manner described herein for device 102 and associated device 104 are further illustrated in FIGS. 3A-3B.

Transducer circuitry 110 includes one or more ultrasound transducer(s) 112 controlled by control circuitry 114. In some embodiments, transducer(s) 112 may include one or more capacitive micromachined ultrasound transducers (CMUTs). CMUTs typically transmit and/or receive ultrasound energy by converting charge stored on a capacitive portion of the CMUT into ultrasound waves carrying ultrasound signals. However, it should be appreciated that transducer(s) 112 may alternatively be formed of piezoelectric material such as PZT. Piezoelectric materials typically transmit and/or receive ultrasound energy by vibrating in accordance with applied electrical voltage at a particular frequency to create ultrasound waves carrying ultrasound signals.

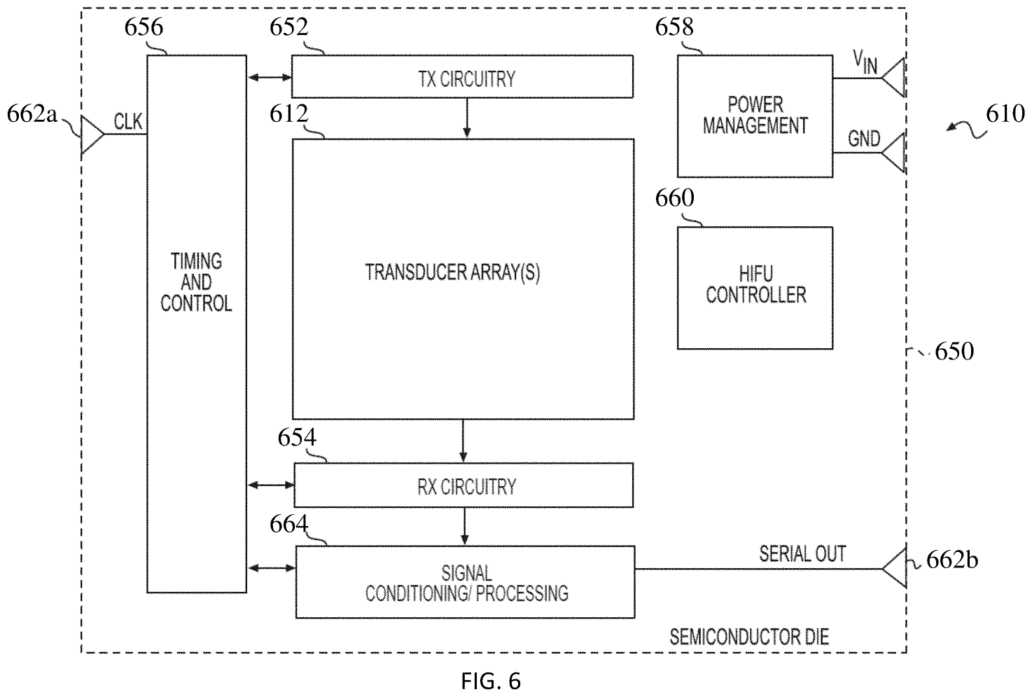

Control circuitry 114 may control transducer(s) 112, such as by driving transducer(s) 112 with pulses to be transmitted to an imaging target. To this end, control circuitry 114 may include one or more programmable waveform generators coupled to one or more pulsers (e.g., multi-level pulsers) to provide electrical signals for driving transducer(s) 112. Control circuitry 114 may also receive ultrasound signals from transducer(s) 112. For instance, ultrasound imaging typically includes transmitting one or more ultrasound pulses towards a target and receiving ultrasound signals reflected back by the target (e.g., echo signals). Accordingly, in embodiments which employ one or more CMUTs, control circuitry 114 may include one or more trans-impedance amplifiers (TIAs) to convert charge on the CMUT(s) into electrical signals. In addition, control circuitry 114 may include one or more analog to digital converters (ADCs) to convert analog signals from transducer(s) 112 into digital signals suitable for processing digitally. In some embodiments, transducer(s) 112 and control circuitry 114 of transducer circuitry 110 may be formed on a single semiconductor die, as described further herein including with reference to FIGS. 6-9.

Power supply 130 may include a printed circuit board having mounted thereon circuitry for converting power from power source 140 such that it may be suitable for operating components of control circuitry 114, processing circuitry 120, and/or transducer(s) 112. For example, power supply 130 may include one or more DC voltage regulators and/or filters. Power source 140 may include one or more batteries, such as rechargeable batteries.

Processing circuitry 120 may include one or more digital logic components and/or one or more radio modules for transmitting ultrasound data wirelessly. For instance, processing circuitry 120 may include one or more logic units such as field programmable gate arrays (FPGAs), application specific integrated circuits (ASICs), and/or microprocessors to transmit the ultrasound data to the radio module for wireless transmission. For instance, the logic units may be configured for interfacing between control circuitry 114 and the radio module(s). In some instances, the logic units may run wireless protocols such as TCP and/or internet protocol (IP). The radio module (e.g., WiFi chip) may include a digital to analog converter (DAC) as well as components for channel coding, mixing, amplifying, and/or beamforming the wireless ultrasound data for transmission via one or more on board or external antennas. It should be appreciated that the antenna(s) may be integrated with the radio module(s) or may be included separately.

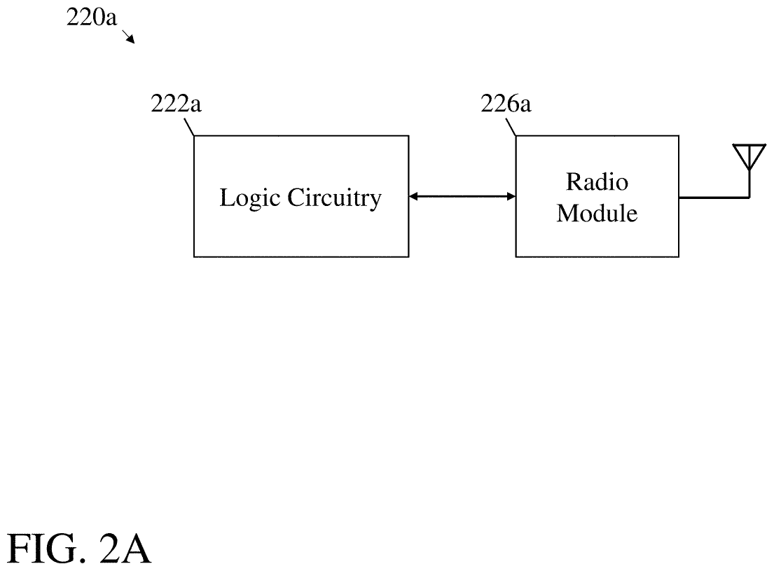

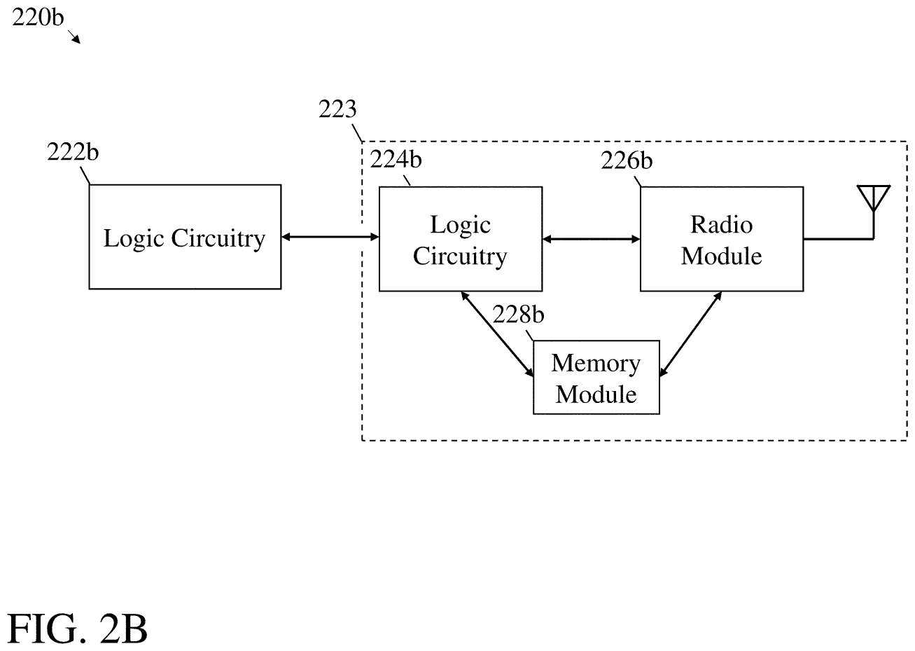

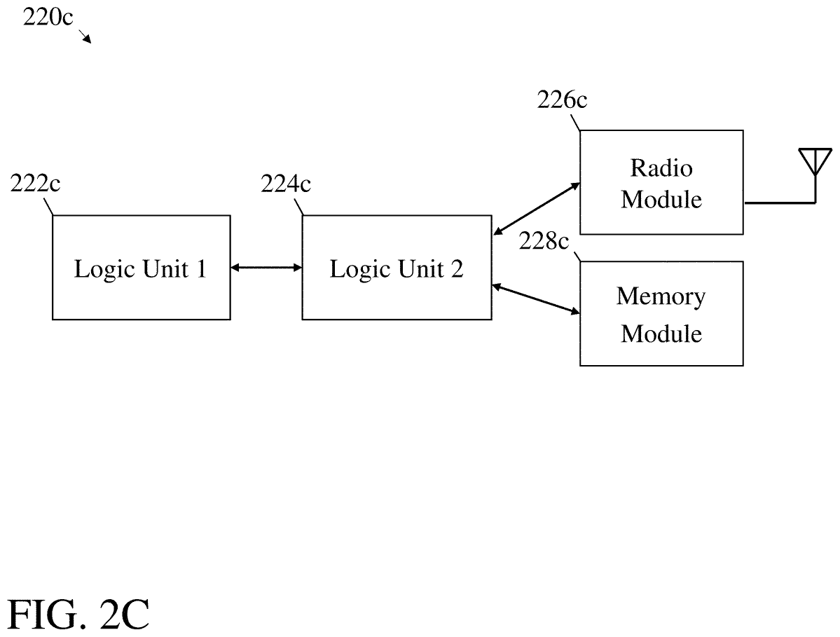

In some embodiments, processing circuitry 120 is integrated within ultrasound device 102. For example, ultrasound device 102 may include one or more logic units and one or more radio modules. In some embodiments, ultrasound device 102 may be configured for removably coupling (e.g., via a USB port) to an auxiliary module carrying a radio module. In some embodiments, the auxiliary module further includes one or more additional logic units configured to receive the ultrasound data from one or more logic units of ultrasound device 102. Processing circuitry 120 is described further herein, including with reference to the exemplary embodiments of FIGS. 2A-2C.

In accordance with various embodiments, associated device 104 may be a computer, a tablet computer, a mobile phone, a wearable device such as a smart watch, or any other suitable computing device capable of receiving wireless data transmission. For instance, associated device may be a laptop computer with an antenna and a wireless network controller configured to receive ultrasound data via a WiFi or Bluetooth connection to device 102. Alternatively, associated device may be a mobile phone configured to receive the ultrasound data via WiFi or Bluetooth. In any case, associated device 104 may include a display suitable for displaying ultrasound images based on ultrasound signals received by transducer(s) 112 and transmitted wirelessly via processing circuitry 120. Alternatively or additionally, device 102 may include a display for displaying such ultrasound images.

FIG. 2A is a block diagram illustrating an exemplary embodiment of processing circuitry 220a for wireless transmission of ultrasound data, in accordance with some embodiments of the technology described herein. Processing circuitry 220a includes logic circuitry 222a and radio module 226a. It should be appreciated that processing circuitry 120 of FIG. 1 may be implemented by processing circuitry 220a in some embodiments of system 100. For instance, logic circuitry 222a may be coupled to transducer circuitry 110 and may receive power from power supply 130. Processing circuitry 220a may be configured to process and wirelessly transmit ultrasound data received from transducer circuitry 110 to associated device 104, in accordance with the embodiment of FIG. 1. In some embodiments, processing circuitry 220a may include one or more printed circuit boards (PCBs), with logic circuitry 222a and/or radio module 226a in the form of one or more integrated circuits (ICs) mounted to substrate(s) of the PCB(s).

Logic circuitry 222a may include one or more logic units configured to receive ultrasound data from the transducer circuitry. For example, the ultrasound data may include ultrasound signals received by the transducer(s) and converted to digital signals via one or more ADCs. The ultrasound signals may be aggregated to form one or more ultrasound images once wirelessly transmitted by processing circuitry 220a to an associated device. To this end, logic circuitry 222a may be configured to prepare the ultrasound data for wireless transmission via radio module 226a. For example, logic circuitry 222a may be configured to run network protocols such as TCP/IP, allocate wireless bandwidth for particular chunks of ultrasound data, or other such tasks.

The logic unit(s) of logic circuitry 222a may be implemented in hardware, such as individual or groups of digital logic gates arranged and/or programmed to perform, based on one or more input signals, various digital logic operations to generate one or more output signals. In some instances, the individual or groups of digital logic gates may be implemented by one or more processors, such as RISC processors, programmed to execute software instructions. In some embodiments, the logic unit(s) of logic circuitry 222a may be FPGA(s). According to non-limiting examples, the FPGA(s) may include an Altera or Microsemi FPGA, and/or an Artix 7 FPGA by Xilinx. The inventors have recognized that FPGAs can be suitable for use with an ultrasound device because FPGAs can be periodically reprogrammed, such as to provide enhanced functionality and/or to be adapted for particular applications. As an example, the FPGA(s) of processing circuitry 220a may be updated to accommodate newly released WiFi standards, such that the wireless ultrasound device can leverage the latest WiFi standards, including new features afforded by the new standards. Alternatively or additionally, in some embodiments the logic unit(s) may be ASICs. The inventors have recognized that ASICs may facilitate implementing wireless ultrasound devices at a low production cost. Alternatively or additionally, in some embodiments the logic unit(s) may be one or more processors. For example, the logic unit(s) may be reduced instruction set computing (RISC) processors. The RISC processor(s) may be configured to run a Unix-based operating system. As a non-limiting example, the logic unit(s) may include an advanced RISC machine (ARM) processor such as an ARMS core processor configured to run Linux. The inventors have recognized that processors can provide a wide range of functions accessible, for example, by associating the logic unit(s) with memory on which software may be temporarily or permanently stored. It should be appreciated that, although not shown in FIG. 2A, processing circuitry 220a may include one or more memory modules such as random access memory (RAM), read only memory (ROM), and/or a port for removably coupling to an external memory device (e.g., a USB port or an expansion card slot).

Radio module 226a may be configured to wirelessly transmit ultrasound data received from logic circuitry 222a to one or more associated devices. For example, radio module 226a may include a DAC as well as components for channel coding, mixing, amplifying, and/or beamforming the wireless ultrasound data for transmission via one or more antennas. The antenna(s) may be formed integrally with radio module 226 or external to radio module 226a.

In some embodiments, radio module 226a may be in the form of an IC mounted to the same substrate of the PCB(s) as logic circuitry 222a. Traces on the substrate may connect radio module 226a to logic circuitry 222a. In some embodiments, radio module 226a and logic circuitry 222a may be mounted to separate substrates. For example, the boards may be coupled to one another by electrical connectors on separate PCBs.

FIG. 2B is a block diagram illustrating an exemplary embodiment of processing circuitry 220b for a wireless ultrasound device removably coupled to auxiliary component 223, in accordance with some embodiments of the technology described herein. Processing circuitry 220b includes logic circuitry 222b, which is removably coupled to auxiliary component 223. It should be appreciated that processing circuitry 120 of FIG. 1 may be implemented by processing circuitry 220b in some embodiments of system 100. For instance, logic circuitry 222b may be coupled to transducer circuitry 110 and may receive power from power supply 130. Processing circuitry 220b may be configured to process and pass ultrasound data received from transducer circuitry 110 to auxiliary component 223, which may be configured to wirelessly transmit the ultrasound data to associated device 104, in accordance with the embodiment of FIG. 1. In some embodiments, processing circuitry 220b may include a PCB with logic circuitry 222b in the form of one or more ICs mounted to the PCB. Auxiliary component 223 may be removably coupled to the PCB, such as by an electrical connector, expansion card slot, or other suitable removable connection.

Logic circuitry 222b may be configured to operate in the manner described for logic circuitry 222a in connection with FIG. 2A. For example, logic circuitry 222b may include one or more ICs mounted on a PCB. However, logic circuitry 222b may be configured for removably coupling to auxiliary module 223. For example, logic circuitry 222b may include a peripheral controller such as a USB controller. As a non-limiting example, logic circuitry 222b may include a Cypress EZ-USB FX3 peripheral controller, which may communicate with an FPGA of logic circuitry 222b via general programmable interface (GPIF). The USB controller may include a USB port configured to receive a USB connector of auxiliary module 223. Alternatively or additionally, logic circuitry 222b may include a slot in which to receive auxiliary module 223. As a non-limiting example, the slot may be an expansion card slot such as a secure digital (SD) slot configured to receive an SD card. An FPGA of logic circuitry 222a may be configured to communicate with auxiliary module 223 using an interface protocol, such as secure digital input output (SDIO) while auxiliary module 223 is received in the slot.

In FIG. 2B, auxiliary module 223 includes logic circuitry 224b, radio module 226b, and memory module 228b. In some embodiments, auxiliary module 223 may include a PCB. The PCB may be contained in a plastic housing with an exposed portion of the board containing electrical contacts for coupling to logic circuitry 222b. In some instances, the exposed portion of the board may terminate in an electrical connector configured for removably coupling to an electrical connector of logic circuitry 222b. As a non-limiting example, auxiliary module 223 may be configured as a USB compatible dongle, such as including a Variscite DART-6UL, which includes an ARM core processor, a CYW43353 802.11c/a/b/g/n radio module, and on-board memory, and which may connect to a USB port of logic circuitry 222b. As a further non-limiting example, auxiliary module 223 may include an SD card, such as a Silex SDCAC which includes a QCA9377 SoC. The inventors have recognized that providing auxiliary module 223 facilitates periodically updating processing circuitry 220b to accommodate new wireless standards. For example, in some embodiments, a wireless ultrasound device can be configured such that the wireless ultrasound device can be opened and auxiliary module 223 may be removed and replaced with a different auxiliary module 223, and one or more FPGA(s) of logic circuitry 222b may be updated to accommodate the different auxiliary module 223. In other embodiments, auxiliary module 223 may be unplugged from an external port of the wireless ultrasound device, such that the device need not be opened for auxiliary module 223 to be replaced with an updated device. As a result, wireless ultrasound devices described herein may be easily updated to leverage the latest wireless standard as new standards are developed (e.g., such that the ultrasound device and/or the auxiliary module 223 do not become outdated due to being programmed according to a particular wireless standard when originally produced). Logic circuitry 224b may include one or more processors such as RISC processors. In a non-limiting example, logic circuitry 224b may include an ARM core processor. It should be appreciated that, in accordance with some embodiments, such as those which implement auxiliary module 223 as an expansion card, processing circuitry 220b may not include logic circuitry 224b and/or memory module 228b. Radio module 226b may be configured to operate in the manner described for radio module 226a in connection with FIG. Memory module 228b may include one or more RAM modules, ROM modules, or other suitable forms of temporary or permanent memory. In some embodiment's, memory 228b may include software to be executed by logic circuitry 224b such as network protocols and/or operating system data.

FIG. 2C is a block diagram illustrating an exemplary embodiment of processing circuitry 220c for wireless transmission of ultrasound data including multiple logic units, in accordance with some embodiments of the technology described herein. In the illustrative embodiment of FIG. 2C, processing circuitry 220c includes first logic unit 222c, second logic unit 224c, radio module 226c, and memory module 228c. It should be appreciated that processing circuitry 120 of FIG. 1 may be implemented by processing circuitry 220c in some embodiments of system 100. For instance, first logic unit 222c may be coupled to transducer circuitry 110 and may receive power from power supply 130. Processing circuitry 220c may be configured to process and wirelessly transmit ultrasound data received from transducer circuitry 110 to associated device 104, in accordance with the embodiment of FIG. 1. First logic unit 222c, second logic unit 224c, radio module 226c, and/or memory module 228c may each be an IC or multiple ICs mounted to one or more PCBs.

First and second logic units 222c and 224c may be implemented in hardware, such as individual or groups of digital logic gates arranged and/or programmed to perform, based on one or more input signals, various digital logic operations to generate one or more output signals. In some instances, the individual or groups of digital logic gates may be implemented by one or more processors, such as RISC processors, programmed to execute software instructions. First logic unit 222c may include one or more FPGA(s). According to non-limiting examples, the FPGA(s) may include an Altera or Microsemi FPGA, and/or an Artix 7 FPGA by Xilinx. Alternatively or additionally, first logic unit 222c may include one or more ASICs. Second logic unit 224c may include one or more processors such as fully functional microprocessors or reduced instruction set computing (RISC) processors. In some embodiments, RISC processor(s) may be configured to run a Unix-based operating system. As a non-limiting example, second logic unit 224c may include an advanced RISC machine (ARM) processor such as an ARMS core processor configured to run Linux. In some embodiments, first and second logic units 222c and 224c may communicate with one another using GPIF. In some embodiments, first logic unit 222c may receive ultrasound data from one or more transducer circuits and second logic unit 224c may perform operations to transmit the ultrasound data wirelessly via radio module 226c. In some embodiments, first logic unit 222c may be implemented by FPGAs and/or ASICs and second logic unit 224c may be implemented by one or more processors such as RISC processors.

Memory module 228c may include one or more RAM modules, ROM modules, or other suitable forms of temporary or permanent memory. In some embodiments, memory 228c may include software to be executed by second logic unit 224c, such as network protocols and/or operating system data. Radio module 226c may be configured to operate in the manner described for radio modules 226a and 226b in connection with FIGS. 2A-2B. As a non-limiting example, radio module 226c may include a Murata 1DX 802.11b/g/n with Bluetooth.

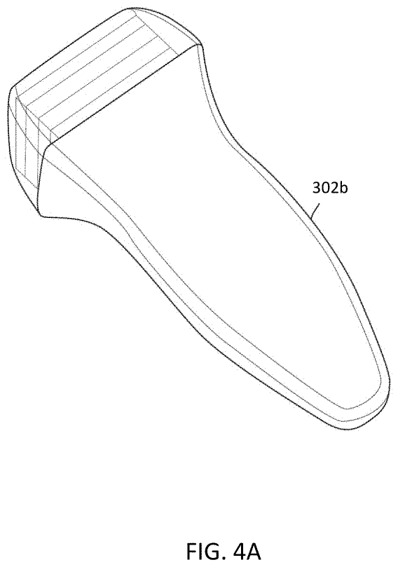

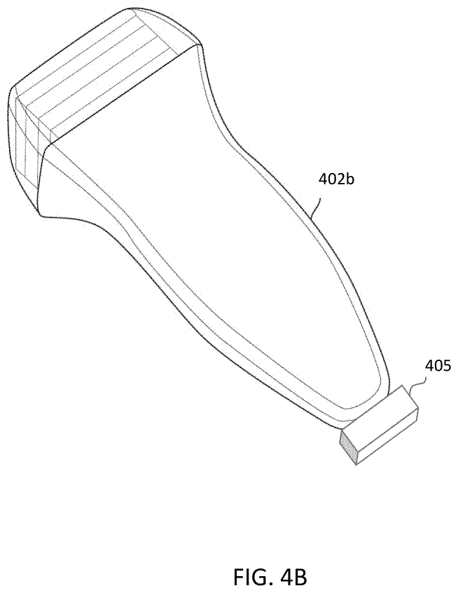

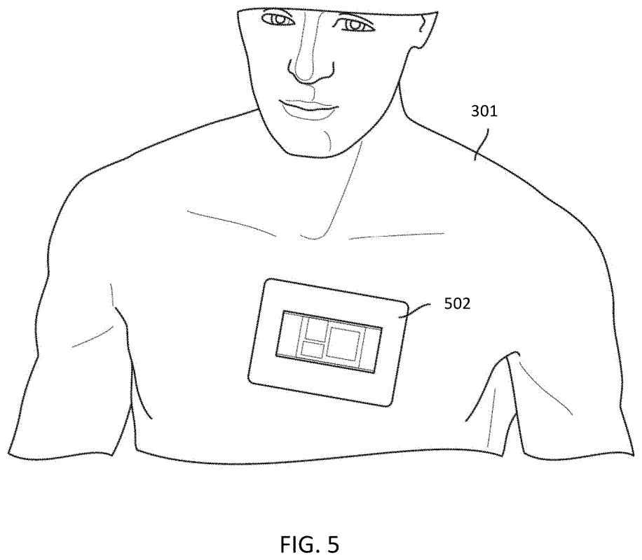

A wireless ultrasound device may be implemented in any of a variety of physical configurations including as part of a handheld device (e.g., discussed in conjunction with FIGS. 3A and 4A-4B), as a part of a pill to be swallowed by a subject (e.g., discussed in conjunction with FIG. 3B), or as part of a patch configured to be affixed to the subject (e.g., discussed in conjunction with FIG. 5).

FIG. 3A is a diagram of exemplary wireless ultrasound system 300a illustrating how handheld wireless ultrasound device 302a may be used to image subject 301 and communicate with associated device 304, in accordance with some embodiments of the technology described herein. For example, although not shown in FIG. 3A, handheld device 302a may include transducer circuitry, processing circuitry, a power supply, and a power source as described in connection with FIG. 1. Further, associated device 304, which is wirelessly coupled to ultrasound device 302a via connection 103, may be configured in the manner described for associated device 104 in connection with FIG. 1. In the illustrative embodiment of FIG. 3A, associated device 304 is a laptop computer. In some embodiments, handheld device 302a includes a screen to display images generated by associated device 304 based on imaging performed by handheld device 302a.

Handheld device 302a may be held against (or near) subject 301 and used to image subject 301. Handheld device 302a may comprise an ultrasound probe. In some embodiments, handheld device 302a further includes a display, which could be a touchscreen display. The display may be configured to display images of the subject generated within handheld device 302a using ultrasound data gathered by the ultrasound probe within handheld device 302a.

In some embodiments, handheld device 302a may be used in a manner analogous to a stethoscope. A medical professional may place handheld device 302a at various positions along a patient's body. The ultrasound probe within handheld device 302a may image the patient. The data obtained by the ultrasound probe may be processed and used to generate image(s) of the patient, which image(s) may be displayed to the medical professional via a display. As such, a medical professional could carry hand-held device 302a (e.g., around their neck or in their pocket) rather than carrying around multiple conventional probes, which is burdensome and impractical.

As shown in FIG. 3A, handheld device 302a is being used to image subject 301. During imaging of subject 301, one or more transducer(s) (not shown) in handheld device 302a may be configured to image subject 301 at or about point 309, located at a depth D2 (e.g., 15-20 cm) from the skin of subject 301. Alternatively or additionally, the transducer(s) in handheld device 302a may be configured to image subject 301 at or about point 307, located at a depth Di (e.g., 1-5 cm) from the skin of subject 301.

In some embodiments, a wireless ultrasound device may be embodied in a pill to be swallowed by a subject. As the pill travels through the subject, the ultrasound probe within the pill may image the subject and wirelessly transmit obtained data to one or more associated devices for processing the data received from the pill and generating one or more images of the subject.

FIG. 3B is a diagram of exemplary wireless ultrasound system 300b illustrating how wireless ultrasound pill 302b may communicate with associated device 304, in accordance with some embodiments of the technology described herein. In the illustrative embodiment of FIG. 3B, device 302b is a wireless ultrasound pill, which may be configured in the manner described for wireless ultrasound device 102. For example, although not shown in FIG. 3B, pill 302b may include transducer circuitry, processing circuitry, a power supply, and a power source, such as those described in connection with FIG. 1.

Pill 302b may be configured to wirelessly transmit ultrasound data to one or more associated devices for further processing. For example, as shown in FIG. 3B, ultrasound pill 302b may be configured to transmit data collected by the transducer(s) on board pill 302b via connection 303 to device 304 (a laptop computer in this non-limiting example), which may process the data to generate and display an image of subject 301 on a display thereof. Associated device 304 may be a desktop, a laptop, a handheld computing device, and/or any other device external to pill 302b, and may be configured to process data received from pill 302b. A person may swallow pill 302b and, as pill 302b travels through the person's digestive system, pill 302b may image the person from within and transmit data obtained by the ultrasound probe within the pill to associated device 304 for further processing.

In some embodiments a wireless ultrasound pill may be implemented by disposing the ultrasound probe within an outer case. In some embodiments, a pill comprising an ultrasound probe may be implemented by encasing the ultrasound probe within an outer housing. In some embodiments, the ultrasound probe that is implemented as part of a pill may comprise one or multiple ultrasonic transducer (e.g., CMUT) arrays, one or multiple image reconstruction chips, processing circuitry including one or more logic units (e.g., FPGAs), communications circuitry, and a power source (e.g., one or more batteries). In some embodiments, the pill may further include a radio module (e.g., WiFi chip).

FIG. 4A illustrates a perspective view of handheld wireless ultrasound device 304a. In some embodiments, handheld device 304a may include processing circuitry 220a, 220b, and/or 220c, such as described in accordance with the embodiments of FIGS. 2A-2C. For example, processing circuitry 220a, 220b, and/or 220c may be contained within a housing of handheld device 304a. In the case of processing circuitry 220b and auxiliary component 223, auxiliary component 223 may be contained within the housing of handheld device 304a such that auxiliary component 223 may be removed from handheld device 304a by opening the housing. In some embodiments, the housing may include a door which may swing (e.g., on a hinge) or which may be removed from the housing (e.g., snapped on or off) to access auxiliary component 223.

In some embodiments, an auxiliary component may removably couple to an external port of a wireless ultrasound device. FIG. 4B illustrates an alternative embodiment of a handheld wireless ultrasound device and an auxiliary component in accordance with the embodiment of FIG. 2B. In FIG. 4B, handheld wireless ultrasound device 402b is removably coupled to auxiliary component 405. Handheld device 402b may be configured to operate in the manner described for handheld device 302a. Auxiliary component 405 may be configured to operate in the manner described for auxiliary component 223. In the illustrative embodiment, auxiliary component 405 may be decoupled from handheld device 402b without opening handheld device 402b. For example, auxiliary component 405 may include an electrical connector to be received in a complementary electrical connector of handheld device 402b. In a non-limiting example, auxiliary component 405 may include a USB connector and handheld device 402b may include a USB port, such that processing circuitry on board handheld device 402a communicates with auxiliary component 405 via USB when auxiliary component 405 is coupled to handheld device 402b. It should be appreciated that, in some embodiments, handheld device 402b may include a USB connector and auxiliary component 405 may include a USB port configured to receive the USB connector. Furthermore, handheld device 402b and auxiliary component 405 may be removably coupled by an electrical cable such as a USB cable.

In some embodiments, a wireless ultrasound probe may be embodied in a patch that may be coupled to a patient. FIG. 5 illustrates wireless ultrasound patch 502, in accordance with some embodiments of the technology described herein. It should be appreciated that patch 502 may be configured in the manner described for wireless ultrasound device 102. For example, although not shown in FIG. 5, patch 502 includes transducer circuitry, processing circuitry, a power supply, and a power source, such as those described in connection with FIG. 1. Patch 502 may be configured to wirelessly transmit data collected by one or more transducers of patch 502 to one or more associated devices (e.g., associated device 304) for further processing.

In some embodiments, patch 502 may include processing circuitry 220a, 220b, and/or 220c in accordance with the embodiments of FIGS. 2A-2C. For example, processing circuitry 220a, 220b, and/or 220c may be contained within a housing of patch 502. The housing may be contained within a dressing used to adhere patch 502 to a patient's skin. In the case of processing circuitry 220b and auxiliary component 223, auxiliary component 223 may be contained within the housing of handheld device 304a such that auxiliary component 223 may be removed from handheld device 304a by opening removing the dressing and opening the housing.

It should be appreciated that, although not illustrated in FIG. 5, in some embodiments, an auxiliary component may removably couple to an external port of patch 502. For example, the port may be exposed through the dressing of patch 502 such that the auxiliary component may be coupled and decoupled to and from patch 502 without needing to remove patch 502 from the patient. In a non-limiting example, the auxiliary component may include a USB connector and patch 502 may include a USB port, such that processing circuitry on board patch 502 communicates with the auxiliary component via USB when the auxiliary component is coupled to patch 502. It should be appreciated that, in some embodiments, patch 502 may include a USB connector and the auxiliary component may include a USB port configured to receive the USB connector. Furthermore, patch 502 and the auxiliary component may be removably coupled by an electrical cable such as a USB cable.

FIG. 6 shows an illustrative example of a monolithic ultrasound device 610 embodying various aspects of the technology described herein. It should be appreciated that, transducer circuitry 110 of FIG. 1 may be implemented as device 610 in accordance with some embodiments of system 100. As shown, the device 610 may include one or more transducer arrangements (e.g., arrays) 612, transmit (TX) circuitry 652, receive (RX) circuitry 654, a timing & control circuit 656, a signal conditioning/processing circuit 664, a power management circuit 658, and/or a high-intensity focused ultrasound (HIFU) controller 660. In the embodiment shown, all of the illustrated elements are formed on a single semiconductor die 650. It should be appreciated, however, that in alternative embodiments one or more of the illustrated elements may be instead located off-chip. In addition, although the illustrated example shows both TX circuitry 652 and RX circuitry 654, in alternative embodiments only TX circuitry or only RX circuitry may be employed. For example, such embodiments may be employed in a circumstance where one or more transmission-only devices 610 are used to transmit acoustic signals and one or more reception-only devices 610 are used to receive acoustic signals that have been transmitted through or reflected off of a subject being ultrasonically imaged.

It should be appreciated that communication between one or more of the illustrated components may be performed in any of numerous ways. In some embodiments, for example, one or more high-speed busses (not shown), such as that employed by a unified Northbridge, may be used to allow high-speed intra-chip communication or communication with one or more off-chip components.

The one or more transducer arrays 612 may take on any of numerous forms, and aspects of the present technology do not necessarily require the use of any particular type or arrangement of transducer cells or transducer elements. Indeed, although the term “array” is used in this description, it should be appreciated that in some embodiments the transducer elements may not be organized in an array and may instead be arranged in some non-array fashion. In various embodiments, each of the transducer elements in the array 612 may, for example, include one or more capacitive micromachined ultrasonic transducers (CMUTs), one or more CMOS ultrasonic transducers (CUTs), one or more piezoelectric micromachined ultrasonic transducers (PMUTs), and/or one or more other suitable ultrasonic transducer cells. In some embodiments, the transducer elements of the transducer array 612 may be formed on the same chip as the electronics of the TX circuitry 652 and/or RX circuitry 654 or, alternatively integrated onto the chip having the TX circuitry 652 and/or RX circuitry 654. In still other embodiments, the transducer elements of the transducer array 612, the TX circuitry 652 and/or RX circuitry 654 may be tiled on multiple chips. The transducer arrays 612, TX circuitry 652, and RX circuitry 654 may be, in some embodiments, integrated in a single ultrasound probe.

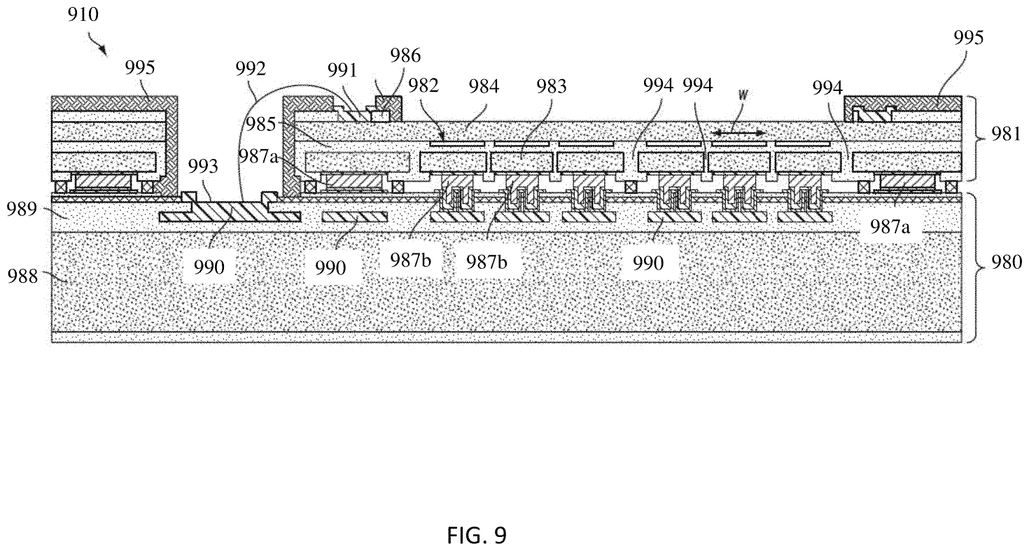

A CUT may include, for example, a cavity formed in a CMOS wafer, with a membrane overlying the cavity, and in some embodiments sealing the cavity. Electrodes may be provided to create a transducer cell from the covered cavity structure. The CMOS wafer may include integrated circuitry to which the transducer cell may be connected. The transducer cell and CMOS wafer may be monolithically integrated, thus forming an integrated ultrasonic transducer cell and integrated circuit on a single substrate (the CMOS wafer). Such embodiments are further described with reference to FIG. 9 below, and additional information regarding microfabricated ultrasonic transducers may also be found in U.S. Pat. No. 9,067,779, assigned to the assignee of the present application, the contents of which are incorporated by reference herein in their entirety.

The TX circuitry 652 (if included) may, for example, generate pulses that drive the individual elements of, or one or more groups of elements within, the transducer array(s) 612 so as to generate acoustic signals to be used for imaging. The RX circuitry 654, on the other hand, may receive and process electronic signals generated by the individual elements of the transducer array(s) 612 when acoustic signals impinge upon such elements.

In some embodiments, the timing & control circuit 656 may be, for example, responsible for generating all timing and control signals that are used to synchronize and coordinate the operation of the other elements in the device 610. In the example shown, the timing & control circuit 656 is driven by a single clock signal CLK supplied to an input port 662a. The clock signal CLK may be, for example, a high-frequency clock used to drive one or more of the on-chip circuit components. In some embodiments, the clock signal CLK may, for example, be a 1.5625 GHz or 2.S GHz clock used to drive a high-speed serial output device in the signal conditioning/processing circuit 664, or a 20 Mhz, 40 MHz, 100 MHz or 200 MHz clock used to drive other digital components on the die 650, and the timing & control circuit 656 may divide or multiply the clock CLK, as necessary, to drive other components on the die 650. In other embodiments, two or more clocks of different frequencies (such as those referenced above) may be separately supplied to the timing & control circuit 656 from an off-chip source.

The power management circuit 658 may be, for example, responsible for converting one or more input voltages VIN from an off-chip source into voltages needed to carry out operation of the chip, and for otherwise managing power consumption within the device 610. In some embodiments, for example, a single voltage (e.g., 1.5 V, 5V, 12V, 80V, 100V, 660V, etc.) may be supplied to the chip and the power management circuit 658 may step that voltage up or down, as necessary, using a charge pump circuit or via some other DC-to-DC voltage conversion mechanism. In other embodiments, multiple different voltages may be supplied separately to the power management circuit 658 for processing and/or distribution to the other on-chip components.

As shown in FIG. 6, in some embodiments, a HIFU controller 660 may be integrated on the die 650 so as to enable the generation of HIFU signals via one or more elements of the transducer array(s) 612. In other embodiments, a HIFU controller for driving the transducer array(s) 612 may be located off-chip, or even within a device separate from the device 610. That is, aspects of the present disclosure relate to provision of ultrasound-on-a-chip HIFU systems, with and without ultrasound imaging capability. It should be appreciated, however, that some embodiments may not have any HIFU capabilities and thus may not include a HIFU controller 660.

Moreover, it should be appreciated that the HIFU controller 660 may not represent distinct circuitry in those embodiments providing HIFU functionality. For example, in some embodiments, the remaining circuitry of FIG. 6 (other than the HIFU controller 660) may be suitable to provide ultrasound imaging functionality and/or HIFU, i .e., in some embodiments the same shared circuitry may be operated as an imaging system and/or for HIFU. Whether or not imaging or HIFU functionality is exhibited may depend on the power provided to the system. HIFU typically operates at higher powers than ultrasound imaging. Thus, providing the system a first power level (or voltage level) appropriate for imaging applications may cause the system to operate as an imaging system, whereas providing a higher power level (or voltage level) may cause the system to operate for HIFU. Such power management may be provided by off-chip control circuitry in some embodiments.

In addition to using different power levels, imaging and HIFU applications may utilize different waveforms. Thus, waveform generation circuitry may be used to provide suitable waveforms for operating the system as either an imaging system or a HIFU system. In some embodiments, the system may operate as both an imaging system and a HIFU system (e.g., capable of providing image-guided HIFU). In some such embodiments, the same on-chip circuitry may be utilized to provide both functions, with suitable timing sequences used to control the operation between the two modalities.

In the example shown, one or more output ports 662b may output a high-speed serial data stream generated by one or more components of the signal conditioning/processing circuit 664. Such data streams may be, for example, generated by one or more USB 2.0, 3.0 and 3.1 modules, and/or one or more 1O GB/s, 40 GB/s, or 1OO GB/s Ethernet modules, integrated on the die 650. In some embodiments , the signal stream produced on output port 662b can be fed to a computer, tablet, or smartphone for the generation and/or display of 2-dimensional, 3-dimensional, and/or tomographic images. In embodiments in which image formation capabilities are incorporated in the signal conditioning/processing circuit 664, even relatively low-power devices, such as smartphones or tablets which have only a limited amount of processing power and memory available for application execution, can display images using only a serial data stream from the output port 662b. As noted above, the use of on-chip analog-to-digital conversion and a high-speed serial data link to offload a digital data stream is one of the features that helps facilitate an “ultrasound on a chip” solution according to some embodiments of the technology described herein.

Device 610 such as shown in FIG. 6 may be used in any of a number of imaging and/or treatment (e.g., HIFU) applications, and the particular examples discussed herein should not be viewed as limiting. In one illustrative implementation, for example, an imaging device including an N×M planar or substantially planar array of CMUT elements may itself be used to acquire an ultrasonic image of a subject, e.g., a person's abdomen, by energizing some or all of the elements in the array(s) 612 (either together or individually) during one or more transmit phases, and receiving and processing signals generated by some or all of the elements in the array(s) 612 during one or more receive phases, such that during each receive phase the CMUT elements sense acoustic signals reflected by the subject. In other implementations, some of the elements in the array(s) 612 may be used only to transmit acoustic signals and other elements in the same array(s) 612 may be simultaneously used only to receive acoustic signals. Moreover, in some implementations, a single imaging device may include a P×Q array of individual devices, or a P×Q array of individual N×M planar arrays of CMUT elements, which components can be operated in parallel, sequentially, or according to some other timing scheme so as to allow data to be accumulated from a larger number of CMUT elements than can be embodied in a single device 610 or on a single die 650.

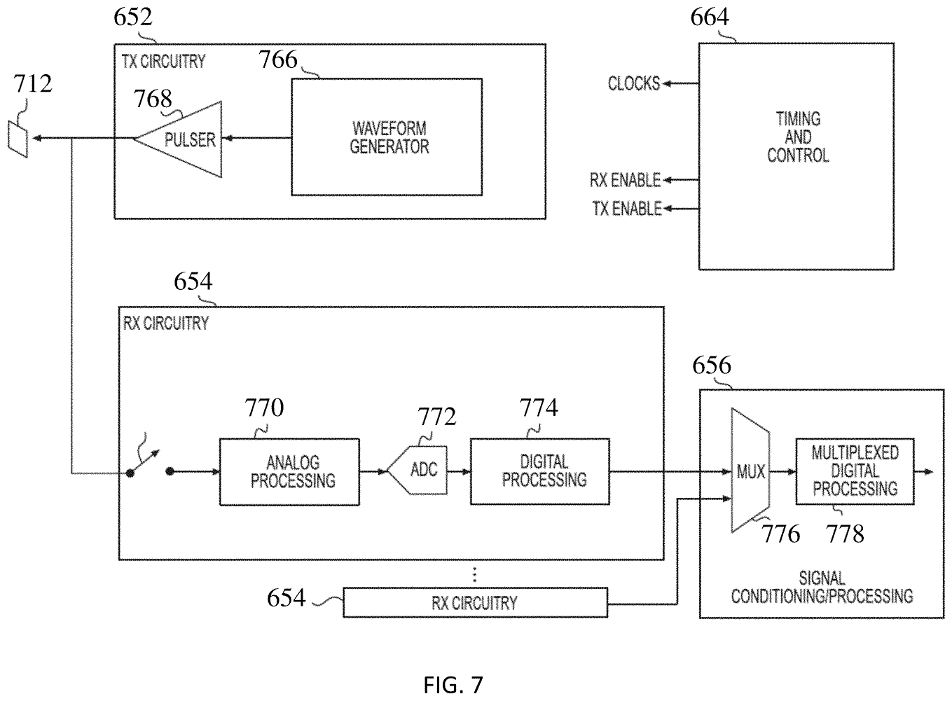

FIG. 7 is a block diagram illustrating how, in some embodiments, the TX circuitry 652 and the RX circuitry 654 for a given transducer element 712 may be used either to energize the transducer element 712 to emit an ultrasonic pulse, or to receive and process a signal from the transducer element 712 representing an ultrasonic pulse sensed by it. In some implementations, the TX circuitry 652 may be used during a “transmission” phase, and the RX circuitry may be used during a “reception” phase that is non-overlapping with the transmission phase. As noted above, in some embodiments, a device 610 may alternatively employ only TX circuitry 652 or only RX circuitry 654, and aspects of the present technology do not necessarily require the presence of both such types of circuitry. In various embodiments, TX circuitry 652 and/or RX circuitry 654 may include a TX circuit and/or an RX circuit associated with a single transducer cell (e.g., a CUT or CMUT), a group of two or more transducer cells within a single transducer element 712, a single transducer element 712 comprising a group of transducer cells, a group of two or more transducer elements 712 within an array 612, or an entire array 612 of transducer elements 712.

In the example shown in FIG. 7, the TX circuitry 652/RX circuitry 654 includes a separate TX circuit and a separate RX circuit for each transducer element 712 in the array(s) 612, but there is only one instance of each of the timing & control circuit 656 and the signal conditioning/processing circuit 664. Accordingly , in such an implementation, the timing & control circuit 656 may be responsible for synchronizing and coordinating the operation of all of the TX circuitry 652/RX circuitry 654 combinations on the die 650, and the signal conditioning/processing circuit 664 may be responsible for handling inputs from all of the RX circuitry 654 on the die 650. In other embodiments, timing and control circuit 656 may be replicated for each transducer element 712 or for a group of transducer elements 712.

As shown in FIG. 7, in addition to generating and/or distributing clock signals to drive the various digital components in the device 610, the timing & control circuit 656 may output either an “TX enable” signal to enable the operation of each TX circuit of the TX circuitry 652, or an “RX enable” signal to enable operation of each RX circuit of the RX circuitry 654. In the example shown, a switch 202 in the RX circuitry 654 may always be opened during the TX circuitry 652 is enabled, so as to prevent an output of the TX circuitry 652 from driving the RX circuitry 654. The switch 202 may be closed when operation of the RX circuitry 654 is enabled, so as to allow the RX circuitry 654 to receive and process a signal generated by the transducer element 712.

As shown, the TX circuitry 652 for a respective transducer element 712 may include both a waveform generator 766 and a pulser 768. The waveform generator 766 may, for example, be responsible for generating a waveform that is to be applied to the pulser 768, so as to cause the pulser 768 to output a driving signal to the transducer element 712 corresponding to the generated waveform.

In the example shown in FIG. 7, the RX circuitry 654 for a respective transducer element 712 includes an analog processing block 770, an analog-to-digital converter (ADC) 772, and a digital processing block 774. The ADC 772 may, for example, comprise an 8-bit, 10-bit, 12-bit or 14-bit, and 5 MHz, 20 MHz, 25 MHz, 40 MHz, 50 MHz, or 80 MHz ADC. The ADC timing may be adjusted to run at sample rates corresponding to the mode based needs of the application frequencies. For example, a 1.5 MHz acoustic signal may be detected with a setting of 20 MHz. The choice of a higher vs. lower ADC rate provides a balance between sensitivity and power vs. lower data rates and reduced power, respectively. Therefore, lower ADC rates facilitate faster pulse repetition frequencies, increasing the acquisition rate in a specific mode.

After undergoing processing in the digital processing block 774, the outputs of all of the RX circuits on the die 650 (the number of which, in this example, is equal to the number of transducer elements 712 on the chip) are fed to a multiplexer (MUX) 776 in the signal conditioning/processing circuit 664. In other embodiments, the number of transducer elements is larger than the number of RX circuits, and several transducer elements provide signals to a single RX circuit. The MUX 776 multiplexes the digital data from the RX circuits, and the output of the MUX 776 is fed to a multiplexed digital processing block 778 in the signal conditioning/processing circuit 664, for final processing before the data is output from the die 650, e.g., via one or more high-speed serial output ports 662b. The MUX 776 is optional, and in some embodiments parallel signal processing is performed. A high-speed serial data port may be provided at any interface between or within blocks, any interface between chips and/or any interface to a host. Various components in the analog processing block 770 and/or the digital processing block 774 may reduce the amount of data that needs to be output from the die 650 via a high-speed serial data link or otherwise. In some embodiments, for example, one or more components in the analog processing block 770 and/or the digital processing block 774 may thus serve to allow the RX circuitry 654 to receive transmitted and/or scattered ultrasound pressure waves with an improved signal-to-noise ratio (SNR) and in a manner compatible with a diversity of waveforms. The inclusion of such elements may thus further facilitate and/or enhance the disclosed “ultrasound-on-a-chip” solution in some embodiments.

Although particular components that may optionally be included in the analog processing block 770 are described below, it should be appreciated that digital counterparts to such analog components may additionally or alternatively be employed in the digital processing block 774.

The converse is also true. That is, although particular components that may optionally be included in the digital processing block 774 are described below, it should be appreciated that analog counterparts to such digital components may additionally or alternatively be employed in the analog processing block 770.

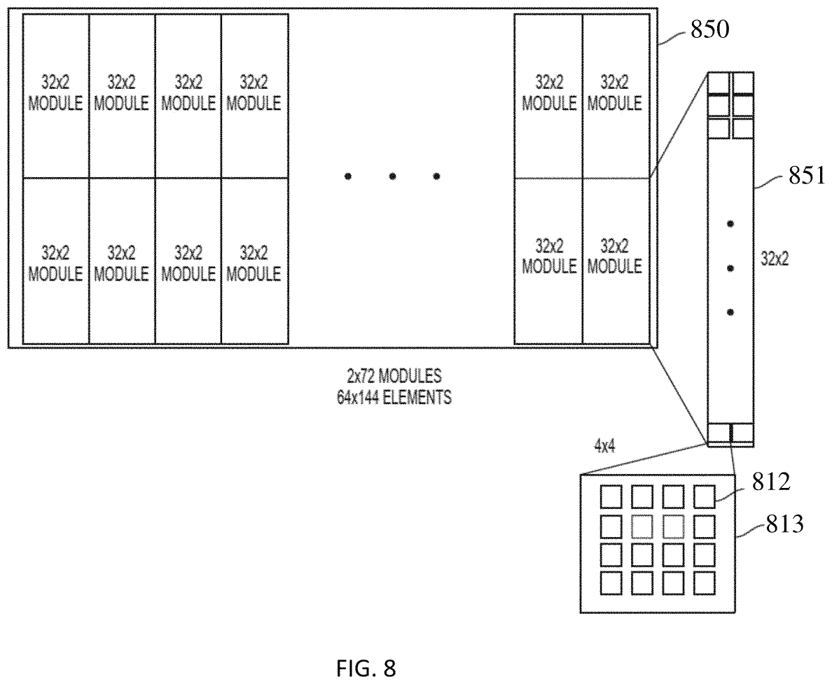

FIG. 8 shows substrate 850 (e.g., a semiconductor die) of an ultrasound device having multiple ultrasound circuitry modules 851 formed thereon. As shown, an ultrasound circuitry module 851 may comprise multiple ultrasound elements 813. An ultrasound element 813 may comprise multiple ultrasonic transducers 812, sometimes termed ultrasonic transducers.

In the illustrated embodiment, substrate 850 comprises 144 modules arranged as an array having two rows and 72 columns. However, it should be appreciated that a substrate of a single substrate ultrasound device may comprise any suitable number of ultrasound circuitry modules (e.g., at least two modules, at least ten modules, at least 100 modules, at least 1000 modules, at least 5000 modules, at least 10,000 modules, at least 25,000 modules, at least 50,000 modules, at least 100,000 modules, at least 250,000 modules, at least 500,000 modules, between two and a million modules, or any number or range of numbers within such ranges) that may be arranged as an two-dimensional array of modules having any suitable number of rows and columns or in any other suitable way.

In the illustrated embodiment, each ultrasound circuitry module 851 comprises 64 ultrasound elements arranged as an array having 32 rows and two columns. However, it should be appreciated that an ultrasound circuitry module may comprise any suitable number of ultrasound elements (e.g., one ultrasound element, at least two ultrasound elements, at least four ultrasound elements, at least eight ultrasound elements, at least 16 ultrasound elements, at least 32 ultrasound elements, at least 64 ultrasound elements, at least 128 ultrasound elements, at least 256 ultrasound elements, at least 512 ultrasound elements, between two and 1024 elements, at least 2500 elements, at least 5,000 elements, at least 10,000 elements, at least 20,000 elements, between 1000 and 20,000 elements, or any number or range of numbers within such ranges) that may be arranged as a two-dimensional array of ultrasound elements having any suitable number of rows and columns or in any other suitable way.

In the illustrated embodiment, each ultrasound element 813 comprises 16 ultrasonic transducers arranged as a two-dimensional array having four rows and four columns. However, it should be appreciated that an ultrasound element may comprise any suitable number and/or groupings of ultrasonic transducer cells (e.g., one, at least two, four, at least four, 9, at least 9, at least 16, 25, at least 25, at least 36, at least 49, at least 64, at least 81, at least 100, between one and 200, or any number or range of numbers within such ranges) that may be arranged as a two dimensional array having any suitable number of rows and columns (square or rectangular) or in any other suitable way. In addition, the transducer cells may include shapes such as circular, oval, square or other polygons, for example.

It should be appreciated that any of the components described above (e.g., ultrasound transmission units, ultrasound elements, ultrasonic transducers) may be arranged as a one-dimensional array, as a two-dimensional array, or in any other suitable manner. In some embodiments, an ultrasound circuitry module may comprise circuitry in addition to one or more ultrasound elements. For example, an ultrasound circuitry module may comprise one or more waveform generators and/or any other suitable circuitry. In some embodiments, module interconnection circuitry may be integrated with the substrate 850 and configured to connect ultrasound circuitry modules to one another to allow data to flow among the ultrasound circuitry modules. For example, the device module interconnection circuitry may provide for connectivity among adjacent ultrasound circuitry modules. In this way, an ultrasound circuitry module may be configured to provide data to and/or received data from one or more other ultrasound circuitry modules on the device.

The ultrasonic transducers of a wireless ultrasound probe may be formed in any of numerous ways and, in some embodiments, may be formed as described with reference to FIG. 9. FIG. 9 is a cross-sectional view of an ultrasound device including a CMOS wafer integrated with an engineered substrate having sealed cavities, according to a non-limiting embodiment of the present application. The device 910 may be formed in any suitable way and, for example, by implementing the methods described in the aforementioned U.S. Pat. No. 9,067,779.

The device 910 includes an engineered substrate 981 integrated with a CMOS wafer 980. The engineered substrate 981 includes a plurality of cavities 982 formed between a first silicon device layer 983 and a second silicon device layer 984. A silicon oxide (Si02) layer 985 (e.g., a thermal silicon oxide-a silicon oxide formed by thermal oxidation of silicon) may be formed between the first and second silicon device layers 983 and 984, with the cavities 982 being formed therein. In this non-limiting example, the first silicon device layer 983 may be configured as a bottom electrode and the second silicon device layer 984 may be configured as a membrane. Thus, the combination of the first silicon device layer 983, second silicon device layer 984, and cavities 982 may form an ultrasonic transducer (e.g., a CMUT), of which six are illustrated in this non-limiting cross-sectional view. To facilitate operation as a bottom electrode or membrane, one or both of the first silicon device layer 983 and second silicon device layer 984 may be doped to act as conductors, and in some cases are highly doped (e.g., having a doping concentration greater than 1015 dopants/cm 3 or greater).

The engineered substrate 981 may further include an oxide layer 986 on top of the second silicon device layer 984, which may represent the BOX layer of a silicon-on-insulator (SOI) wafer used to form the engineered substrate 981. The oxide layer 986 may function as a passivation layer in some embodiments and, as shown, may be patterned to be absent over the cavities 982. Contacts 991, and passivation layer 995 may be included on the engineered substrate 981. The passivation layer 995 may be patterned to allow access to one or more contacts 991, and may be formed of any suitable passivating material. In some embodiments, the passivation layer 995 is formed of silicon nitride Si3N4 and in some embodiments is formed by a stack of Si02 and SbN4, although alternatives are possible.

The engineered substrate 981 and CMOS wafer 980 may be bonded together at bond points 987a and 987b. The bond points may represent eutectic bond points, for example formed by a eutectic bond of a layer on engineered substrate 981 with a layer on CMOS wafer 980, or may be any other suitable bond type described herein (e.g., a silicide bond or thermocompression bond). In some embodiments, the bond points 987a and 987b may be conductive, for example being formed of metal. The bond points 987a may function solely as bond points in some embodiments, and in some embodiments may form a seal ring, for example hermetically sealing the ultrasonic transducers of the device 910, and improving device reliability. In some embodiments, the bond points 987a may define a seal ring that also provides electrical connection between the engineered substrate and CMOS wafer. Similarly, the bond points 987b may serve a dual purpose in some embodiments, for example serving as bond points and also providing electrical connection between the ultrasonic transducers of the engineered substrate 981 and the IC of the CMOS wafer 980. In those embodiments in which the engineered substrate is not bonded with a CMOS wafer the bond points 987b may provide electrical connection to any electrical structures on a substrate to which the engineered substrate is bonded.

The CMOS wafer 980 includes a base layer (e.g., a bulk silicon wafer) 988, an insulating layer 989 (e.g., Si02), and a metallization 990. The metallization 990 may be formed of aluminum, copper, or any other suitable metallization material, and may represent at least part of an integrated circuit formed in the CMOS wafer. For example, metallization 990 may serve as a routing layer, may be patterned to form one or more electrodes, or may be used for other functions. In practice, the CMOS wafer 980 may include multiple metallization layers and/or post-processed redistribution layers, but for simplicity, only a single metallization is illustrated.

The bond points 987b may provide electrical connection between the metallization 990 of CMOS wafer 980 and the first silicon device layer 983 of the engineered substrate. In this manner, the integrated circuitry of the CMOS wafer 980 may communicate with (e.g., send electrical signals to and/or receive electrical signals from) the ultrasonic transducer electrodes and/or membranes of the engineered substrate. In the illustrated embodiments, a separate bond point 987b is illustrated as providing electrical connection to each sealed cavity (and therefore for each ultrasonic transducer), although not all embodiments are limited in this manner. For example, in some embodiments, the number of electrical contacts provided may be less than the number of ultrasonic transducers.

Electrical contact to the ultrasonic transducer membranes represented by second silicon device layer 984 is provided in this non-limiting example by contacts 991, which may be formed of metal or any other suitable conductive contact material. In some embodiments , an electrical connection may be provided between the contacts 991 and the bond pad 993 on the CMOS wafer. For example, a wire bond 992 may be provided or a conductive material (e.g., metal) may be deposited over the upper surface of the device and patterned to form a conductive path from the contacts 991 to the bond pad 993. However, alternative manners of connecting the contacts 991 to the IC on the CMOS wafer 980 may be used. In some embodiments an embedded via (not shown in FIG. 9) may be provided from the first silicon device layer 983 to a bottom side of the second silicon device layer 984, thus obviating any need for the contacts 991 on the topside of the second silicon device layer 984. In such embodiments, suitable electrical isolation may be provided relative to any such via to avoid electrically shorting the first and second silicon device layers.

The device 910 also includes isolation structures (e.g., isolation trenches) 994 configured to electrically isolate groups of ultrasonic transducers (referred to herein as “ultrasonic transducer elements”) or, as shown in FIG. 9, individual ultrasonic transducers. The isolation structures 994 may include trenches through the first silicon device layer 983 that are filled with an insulating material in some embodiments. Alternatively, the isolation structures 994 may be formed by suitable doping. Isolation structures 994 are optional.

Various features of the device 910 are now noted. For instance, it should be appreciated that the engineered substrate 981 and CMOS wafer 980 wafer may be monolithically integrated, thus providing for monolithic integration of ultrasonic transducers with CMOS ICs. In the illustrated embodiment, the ultrasonic transducers are positioned vertically (or stacked) relative to the CMOS IC, which may facilitate formation of a compact ultrasound device by reducing the chip area required to integrate the ultrasonic transducers and CMOS IC.

Additionally, the engineered substrate 981 includes only two silicon layers 983 and 984, with the cavities 982 being formed between them. The first silicon device layer 983 and second silicon device layer 984 may be thin, for example each being less than 50 microns in thickness, less than 30 microns in thickness, less than 20 microns in thickness, less than 10 microns in thickness, less than 5 microns in thickness, less than 3 microns in thickness, or approximately 2 microns in thickness, among other non-limiting examples. In some embodiments it is preferable for one of the two wafers (e.g., silicon layer 983 or silicon layer 984) of the engineered substrate to be sufficiently thick to minimize vibration, prevent vibration or shift the frequency of unwanted vibration to a range outside of the operating range of the device, thereby preventing interference. Through modeling of the geometries in the physical stack of the transducer integrated with the CMOS, thicknesses of all layers can be optimized for transducer center frequency and bandwidth, with minimal interfering vibration. This may include, but is not limited to, changing layer thicknesses and features in the transducer engineered substrate and changing the thickness of the CMOS wafer 988. These layer thicknesses are also chosen to provide uniformity across the area of the array, and therefore tighter frequency uniformity, using commercially available wafers.

Thus, while the engineered substrate may be thin, it may have a thickness of at least, for example, 4 microns in some embodiments, at least 5 microns in some embodiments, at least 7 microns in some embodiments, at least 10 microns in some embodiments, or other suitable thickness to prevent unwanted vibration. Such dimensions contribute to achieving a small device and may facilitate making electrical contact to the ultrasonic transducer membrane (e.g., second silicon device layer 984) without the need for thru-silicon vias (TSVs). TSVs are typically complicated and costly to implement, and thus avoiding use of them may increase manufacturing yield and reduce device cost. Moreover, forming TSVs requires special fabrication tools not possessed by many commercial semiconductor foundries, and thus avoiding the need for such tools can improve the supply chain for forming the devices, making them more commercially practical than if TSVs were used.

The engineered substrate 981 as shown in FIG. 9 may be relatively thin, for example being less than I 00 microns in total thickness, less than 50 microns in total thickness, less than 30 microns in total thickness, less than 20 microns in total thickness, less than 10 microns in total thickness, or any other suitable thickness. The significance of such thin dimensions includes the lack of structural integrity and the inability to perform various types of fabrication steps (e.g., wafer bonding, metallization, lithography and etch) with layers having such initially thin dimensions. Thus, it is noteworthy that such thin dimensions may be achieved in the device 910, via a process sequence.

Also, the silicon device layers 983 and 984 may be formed of single crystal silicon. The mechanical and electrical properties of single crystal silicon are stable and well understood, and thus the use of such materials in an ultrasonic transducer (e.g., as the membrane of a CMUT) may facilitate design and control of the ultrasonic transducer behavior.

In one embodiment, there is a gap between parts of the CMOS wafer 980 and the first silicon device layer 983 since the two are bonded at discrete bond points 4 1 6b rather than by a bond covering the entire surface of the CMOS wafer 980. The significance of this gap is that the first silicon device layer 983 may vibrate if it is sufficiently thin. Such vibration may be undesirable, for instance representing unwanted vibration in contrast to the desired vibration of the second silicon device layer 984. Accordingly, it is beneficial in at least some embodiments for the first silicon device layer 983 to be sufficiently thick to minimize vibration, avoid vibration or shift the frequency of any unwanted vibration outside of the operating frequency range of the device.

In alternative embodiments, it may be desirable for both the first and second silicon device layers 983 and 984 to vibrate. For instance, they may be constructed to exhibit different resonance frequencies, thus creating a multi-frequency device. The multiple resonance frequencies (which may be related as harmonics in some embodiments) may be used, for example, in different operating states of an ultrasonic transducer. For example, the first silicon device layer 983 may be configured to resonate at half the center frequency of the second silicon device layer 984.

In still another embodiment, the strength of the bond between silicon device layer 984 and silicon oxide layer 985 allows for cavities 982 formed within silicon oxide layer 985 to have a larger diameter than would be possible with a weaker bond between layers 984 and 985. The diameter of a cavity is indicated as “w” in FIG. 9. The bond strength is provided at least in part by using a fabrication process in which the engineered substrate 981 is formed by bonding (e.g., at temperature less than about 910° C.) of two wafers, one containing silicon device layer 983 and the other containing silicon device layer 984, followed by a high temperature anneal (e.g., about 1000° C.). Ultrasonic transducers implemented using wide cavities may generate ultrasonic signals having more power at a particular frequency than ultrasonic signals generated at the same particular frequency by ultrasonic transducers implemented using cavities have a smaller diameter. In turn, higher power ultrasonic signals penetrate deeper into a subject being imaged thereby enabling high-resolution imaging of a subject at greater depths than possible with ultrasonic transducers having smaller cavities. For example, conventional ultrasound probes may use high frequency ultrasound signals (e.g., signals having frequencies in the 7-12 MHz range) to generate high-resolution images, but only at shallow depths due to the rapid attenuation of high-frequency ultrasound signals in the body of a subject being imaged. However, increasing the power of the ultrasonic signals emitted by an ultrasound probe (e.g., as enabled through the use of cavities having a larger diameter as made possible by the strength of the bond between layers 984 and 985) allows the ultrasonic signals to penetrate the subject deeper resulting in high-resolution images of the subject at greater depths than previously possible with conventional ultrasound probes.

Additionally, an ultrasonic transducer formed using a larger diameter cavity may generate lower frequency ultrasound signals than an ultrasonic transducer having a cavity with a smaller diameter. This extends the range of frequencies across which the ultrasonic transducer may operate. An additional technique mat be to selectively etch and thin portions of the transducer top membrane 984. This introduces spring softening in the transducer membrane, thereby lowering the center frequency. This may be done on all, some or none of the transducers in the array in any combination of patterns.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

What is claimed is:

1. An ultrasound device, comprising:
one or more ultrasound transducers configured to perform ultrasound imaging;
a first logic unit configured to receive ultrasound data from the one or more ultrasound transducers; and
a second logic unit coupled to the first logic unit and configured to transmit the ultrasound data wirelessly via a radio module.

2. The ultrasound device of claim 1, wherein the first logic unit comprises a first group of digital logic gates.

3. The ultrasound device of claim 2, wherein the first group of digital logic gates comprises a field programmable gate array (FPGA).

4. The ultrasound device of claim 2, wherein the first group of digital logic gates comprises an application specific integrated circuit (ASIC).

5. The ultrasound device of claim 1, wherein the first logic unit and the second logic unit are configured to communicate using a general programmable interface (GPIF) protocol.

6. The ultrasound device of claim 2, wherein the second logic unit comprises a second group of digital logic gates.

7. The ultrasound device of claim 6, wherein the second group of digital logic gates comprises a processor.

8. The ultrasound device of claim 7, wherein the processor comprises a reduced instruction set computing (RISC) processor.

9. The ultrasound device of claim 8, wherein the RISC processor comprises an advanced RISC machine (ARM) processor.

10. The ultrasound device of claim 7, wherein the processor is configured to run an operating system.

11. The ultrasound device of claim 10, wherein the operating system is unix-based.

12. The ultrasound device of claim 7, wherein the processor is configured to manage wireless networking protocols for transmitting the ultrasound data wirelessly via the radio module.

13. The ultrasound device of claim 1, wherein the radio module includes a WiFi module.

14. The ultrasound device of claim 1, further comprising a memory module coupled to the second logic unit.
